(19) United States
(12) Patent Application Publication
(10) Pub. No.: US 2024/0335413 A1
(43) Pub. Date: Oct. 10, 2024

(54) LIP BALM COMPOSITION AND METHOD FOR MAKING AND USING THE SAME

(71) Applicant: Anthony Beretta, Greenwood, IN (US)

(72) Inventor: Anthony Beretta, Greenwood, IN (US)

(21) Appl. No.: 18/295,494

(22) Filed: Apr. 4, 2023

(51) Int. Cl. A61K 31/245; A61K 9/00; A61K 31/045; A61K 31/222; A61K 33/30; A61K 35/64; A61K 36/185; A61K 36/534; A61K 36/54; A61K 47/44

(52) U.S. Cl. CPC A61K 31/245; A61K 9/0014; A61K 31/045; A61K 31/222; A61K 33/30; A61K 35/64; A61K 36/185; A61K 36/534; A61K 36/54; A61K 47/44

(57) ABSTRACT

A method for topically reducing pain and stimulating healing, including providing a homogeneous composition including 15 to 20 weight percent benzocaine, 1 to 2 weight percent menthol, 5 to 15 weight percent zinc oxide, to 10 weight percent octinovate, 1 to 5 weight percent peppermint oil, 0 to 10 weight percent cinnamon oil, 0 to 20 weight percent bee venom, 5 to 20 weight percent cocoa butter, with the balance being nonreactive base matrix material, and then topically applying the homogeneous composition to skin where pain reduction and healing is desired.

### TECHNICAL FIELD

The invention relates generally to the field of cosmetics, and more specifically to a composition for speeding the recovery from cold sores and blemishes and method of using the same.

### BACKGROUND

Skin blisters, blemishes, and aberrations, such as cold sores from oral herpes, welts from insect bites and stings, abrasions and/or cuts can be irritating and take a long time to heal. While many products exist to disinfest the wounds and/or keep the affected skin moist, very few actually promote rapid healing. Current symptomatic treatments include pharmaceutical analgesics, in particular non-steroidal/anti-inflammatory agents such as such as aspirin, ibuprofen, acetaminophen, more powerful analgesics such as codeine and the like, as well as topical gel anesthetics containing lidocaine or benzocaine. However, these pharmaceuticals all have their own inherent limitations, insofar as they may not be available without a prescription, may not have an immediate effect, and/or may have undesirable side effects. There remains a need for improved methods of treating the irritation caused by such skin issues, which both address the various types of pain and irritation as well as promote more rapid healing of the same. The present novel technology addresses this need.

### DETAILED DESCRIPTION OF THE PREFERRED EMBODIMENTS

For the purposes of promoting an understanding of the principles of the claimed technology and presenting its currently understood best mode of operation, reference will now be made to the embodiments and specific language will be used to describe the same. It will nevertheless be understood that no limitation of the scope of the invention is thereby intended, with such alterations and further modifications in the illustrated device and such further applications of the principles of the claimed technology as illustrated therein being contemplated as would normally occur to one skilled in the art to which the claimed technology relates.

The instant novel technology relates to 1 a skin care pain relief and healing stimulant formula. The formula is generally given as follows: 5 to 20 weight percent benzocaine; 1 to 2 weight percent menthol; 5 to 15 weight percent zinc oxide; 5 to 10 weight percent octinovate; 1 to 5 weight percent peppermint oil; 0 to 10 weight percent cinnamon oil; 0 to 20 weight percent bee venom; and 5 to 20 weight percent cocoa butter; with the balance being nonreactive base matrix material.

As used herein, cinnamon oil is taken as having a density of 1.03 g/mL at 25° C. and is described by CAS No. 8007-80-5. Peppermint oil is taken as having a density of 0.899 g/mL at 25° C., has a menthol content of 56%, and is described by CAS No. 8006-90-4. Menthol essential oil, also known as Mentha arvensis oil, as referenced herein has essentially the same physical properties as peppermint oil. Cocoa butter has a density range of 0.86 to 0.94 g/mL and is taken herein to have a density of 0.90 g/mL.

In one embodiment of the present novel technology, a skin care pain relief and healing stimulant formula is prepared as follows. Active components benzocaine, menthol, zinc oxide, octinovate (all typically provided in solid, powdered format), and peppermint oil are combined and mixed to yield a homogeneous admixture, according to the relative amounts given in Table 1, with the balance being an inert and/or nonreactive base matrix material, in this example a petrolatum base. One or two drops of the resulting admixture (or, in the case wherein the admixture is a solid or semi-solid, a thin coating) is/are applied topically to the affected area once or twice a day, or as needed, to both dull any pain and promote healing. The above admixture may be varied by increasing or decreasing the amount of one or more active ingredients by a factor of up to two, or even up to three or four.

In a second embodiment, the following oils and like components are mixed according to the weights given in Table 2, with the balance again being a petrolatum matrix, to yield a homogeneous admixture. One or two drops of the resulting admixture are applied topically to the affected area once or twice a day, or as needed, to both dull any pain and promote healing. The above admixture may be varied by increasing or decreasing the amount of one or more ingredients by a factor of up to two, or even up to three or four.

TABLE 1

| Active Component | Weight Percent |
|---|---|
| Benzocaine | 20 |
| Menthol | 1 |
| Zinc Oxide | 10 |
| Octinovate | 7.5 |
| Peppermint Oil | 1.5 |
| Cocoa Butter | 10 |
| Petrolatum | balance |

TABLE 2

| Active Component | Weight Percent |
|---|---|
| Benzocaine | 20 |
| Menthol | 1 |
| Zinc Oxide | 10 |
| Octinovate | 7.5 |
| Cinnamon | 10 |
| Peppermint Oil | 1.5 |
| Cocoa Butter | 10 |
| Petrolatum | balance |

In another embodiment, the instant formulation is a combination of constituents as presented in Table 3 and includes the component bee venom. The bee venom is present in concentrations as harvested, and the constituents are well mixed to yield a homogeneous admixture.

TABLE 3

| Active Component | Weight Percent |
|---|---|
| Bee Venom | 15 |
| Benzocaine | 15 |
| Menthol | 2 |
| Zinc Oxide | 10 |
| Octinovate | 8 |
| Peppermint Oil | 2 |
| Cocoa Butter | 10 |
| Base Matrix | balance |

In yet another embodiment, the instant preparation is a combination of constituents as presented in Table 4 and well mixed to yield a homogeneous admixture. The relative amounts of each are typically as presented in Table 4 above, although the amounts of one or more of the above-listed constituents may be varied by half to by double.

TABLE 4

| Active Component | Weight Percent |
|---|---|
| Bee Venom | 10 |
| Benzocaine | 15 |
| Menthol | 2 |
| Zinc Oxide | 10 |
| Octinovate | 8 |
| Cinnamon | 5 |
| Peppermint Oil | 5 |
| Cocoa Butter | 10 |
| Petrolatum | balance |

One or two drops or a thin coating of the resulting admixture may be applied topically to the affected area once or twice a day. Pain relief and/or healing promotion should be observed immediately upon application.

While the invention has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the invention are desired to be protected.

What is claimed is:

1. A method for topically reducing pain and stimulating healing, comprising:
a) providing a homogeneous composition including 20 weight percent benzocaine, 1 weight percent menthol, 10 weight percent zinc oxide, 7.5 weight percent octinovate, 1.5 weight percent peppermint oil, 10 weight percent cocoa butter, with the balance being petrolatum; and
b) topically applying the homogeneous composition to skin where pain reduction and healing is desired.

2. The method of claim 1 wherein step b) is repeated at least twice daily until desired healing and pain reduction is achieved.

3. A pain reduction topical composition comprising in combination:
15 to 20 weight percent benzocaine;
1 to 2 weight percent menthol;
5 to 15 weight percent zinc oxide;
5 to 10 weight percent octinovate;
1 to 5 weight percent peppermint oil;
0 to 10 weight percent cinnamon oil;
0 to 20 weight percent bee venom;
5 to 20 weight percent cocoa butter;
with the balance being nonreactive base matrix material.

4. A topical pain reduction and healing composition comprising in combination:
one part by weight petrolatum base material; and
one part by weight admixture;
wherein the admixture further comprises:
15 to 20 parts by weight benzocaine;
1 to 2 parts by weight menthol;
5 to 15 parts by weight zinc oxide;
5 to 10 parts by weight octinovate;
1 to 5 parts by weight peppermint oil;
0 to 10 parts by weight cinnamon oil;
0 to 20 parts by weight bee venom; and
5 to 20 parts by weight cocoa butter.

5. The topical pain reduction and healing composition of claim 4 wherein the admixture further comprises:
20 parts by weight benzocaine;
1 parts by weight menthol;
10 parts by weight zinc oxide;
7.5 parts by weight octinovate;
1.5 parts by weight peppermint oil;
0 parts by weight cinnamon oil;
0 parts by weight bee venom; and
10 parts by weight cocoa butter.

6. The topical pain reduction and healing composition of claim 4 wherein the admixture further comprises a finite amount of cinnamon oil.

7. The topical pain reduction and healing composition of claim 4 wherein the admixture further comprises a finite amount of bee venom.

8. The topical pain reduction and healing composition of claim 4 wherein the admixture further comprises at least some bee venom and at least some cinnamon oil.
